(19) United States
(12) Patent Application Publication
(10) Pub. No.: US 2008/0306470 A1
(43) Pub. Date: Dec. 11, 2008

(54) Optical screening device

(76) Inventor: Joshua Friedman, Ridgefield, CT (US)

Correspondence Address: Eugene Lieberstein, 2151 Long Ridge Road, Stamford, CT, 06903, US

(21) Appl. No.: 12/151,117

(22) Filed: May 5, 2008

Related U.S. Application Data
(63) Continuation-in-part of application No. 11/811,580, filed on Jun. 11, 2007.

(51) Int. Cl. A61B 1/07; A61B 1/24

(52) U.S. Cl. 606/3

(57) ABSTRACT

An optical screening device for detecting abnormal skin tissue indicative of disease including a handle having an elongated stem adapted for holding the optical screening device in one hand and a head for housing an array of light emitting diodes with each diode in the array being spaced apart to form a given geometry with each diode selected to emit light in a wavelength range between 340 nm to 470 nm; a high pass filter supported by said handle in close proximity to said array of light emitting diodes with the filter being of a size to provide a defined viewable area for visibly detecting irradiated light reflected from skin tissue under observation and adapted to block light at least below 400 nm; and a source of electrical power for energizing said array of light emitting diodes.

### FIELD OF INVENTION

This is a continuation-in-part of U.S. patent application Ser. No. 11/811,580 filed Jun. 11, 2007 to an optical screening device for detecting abnormal skin tissue in a mammal and particularly in the mouth indicative of disease such as cancer or a pre-cancerous oral tissue.

### BACKGROUND

Oral cancer affects over 30,000 people today in the U.S. alone. The treatment for this disease is most effective when diagnosed early. When diagnosed late the treatment is unpleasant, disfiguring and often not effective. Advanced oral cancer is treated with surgery, chemo and radiation much the same as other aggressive cancers.

The early signs of oral cancer are not always discernable upon visual examination. In an attempt to improve the visualization other modalities have been developed to assist in screening for cancer and pre-cancerous lesions. Some of these procedures involve the use of toluidine blue rinse, acetic acid rinse and biofluorescence.

As in all cancers, a biopsy is used to make a positive diagnosis; however the process of taking a biopsy tissue sample is time consuming, costly and painful. It also requires special training and as consequence, most general dentists elect to send patients to an oral surgeon or oral pathologist for a biopsy procedure.

It would be highly desirable, therefore, to have a screening device that is easy to use, low in cost and effective. Such a device is useful even if a biopsy is still necessary provided it could eliminate false positives, permit the unnecessary taking of a biopsy or be indicative of when a biopsy for lesions is likely to be cancer or pre-cancer. Such a device would also save time, money and lives.

In the past, chemoluminesence together with an acetic rinse have been used to detect acetowhite lesion or luekoplakia. This technique has been used in the screening for cervical cancer as well as oral cancer. However, it does not work for all lesions and does not delineate the border between healthy and pre-cancerous or dysplastic or cancerous tissue.

Bioflouresence is also used for detection of pre-oral cancer and oral cancer. This technique for pre-oral cancer and oral cancer detection uses a light source that can typically provide energy in the 340 NM to 470 NM range. It is known to those skilled in the art that within this wavelength distribution, oral tissue will fluoresce, while diseased tissue that exhibits varying degrees of dysplasia (pre cancer) will not fluoresce. It is also known that fluorescence normally takes place at a wavelength 40-60 NM higher than the excitation source or energy. In order to enhance the contrast between healthy and diseased tissue, a filter can be employed to block the excitation source and pass the fluorescent energy.

A device designed to use the technique of bioflouresence for oral cancer screening is currently manufactured by LED Electronics, Vancouver Canada. This device is described in detail in patent application Ser. No. 11/016,567, publication number US 2005/0234526 A1, by Gilhuly and Whitehead and uses a metal halide light source with a fiber optic or liquid light guide together with associated optics and filters. The device described in the Gilhuly patent application has many drawbacks. It is based upon the use of a metal halide lamp, specialized high voltage power supply, bandpass filter, means to block excess heat and unwanted energy, a fan cooling system, fiber optic light guide and specialized means to view the tissue. A metal halide lamp produces a broad range or energy spectrum from below 300 NM to visible light and infra-red energy well above 800 NM. Accordingly, various blocking filters are required to provide useful excitation energy in the required spectrum. Moreover, since a metal halide light source provides most of its energy outside the useful wavelength spectrum for this procedure, this device is complex, unnecessarily large, bulky and extremely expensive.

### SUMMARY OF THE INVENTION

The optical screening device of this invention employs an array of light emitting diodes and a single blocking filter to cause suspected tissue to fluoresce enabling the discrimination between cancer, pre-cancerous, and normal tissue. The array of light emitting diodes may consist of only one interconnected array of light emitting diodes to emit light in a wavelength range of between 340 nm to 400 nm or between 400 nm to 470 nm or may include a first and second array of interconnected light emitting diodes to emit light in a first wavelength range of between 340 nm to 400 nm and in a second wavelength range of between 400 nm to 470 nm. In the latter case it is preferred to also to include a switching means to activate either the first plurality of interconnected light emitting diodes or the second plurality of interconnected light emitting diodes. In another mode of operation, all LED's from 340 to 400 nm and from 400 to 470 nm may be illuminated simultaneously.

The blocking filter provides a screening area for visually observing the suspected skin tissue and should be of a defined size.

### BRIEF DESCRIPTION OF THE DRAWINGS

FIG. 1 is a front view of the device of the present invention showing the array of LED's in the head of the device and showing with dotted lines the internal batteries as the power source for the device;

FIG. 2 is a schematic view in perspective of the device in operation for screening the oral cavity in the mouth of a dental patient;

FIG. 3 is a circuit schematic of one embodiment of a control circuit and voltage regulator for activating the array of LED's in the device of the present invention;

FIG. 4 is a circuit schematic of a second embodiment of a control circuit and voltage regulator for activating the array of LED's in the device of the present invention;

FIG. 5 is a partial view of an alternate embodiment of the present invention having a dual array of LED's in the head of the device;

FIG. 6 is a diagrammatic illustration of a second embodiment of the invention;

FIG. 7 is a graphical illustration of the use of the second embodiment in accordance with the present invention

FIG. 8 is a partial view in perspective of the light source shown in FIG. 6;

FIGS. 9(a)-9(d) show alternate light guides which may be used in the light source shown in FIG. 6 and 8 respectively; and

FIG. 10(a) shows a metal clip for securing the light guide in the light source shown in FIG. 6 and 8 respectively;

FIG. 10(b) shows an o-ring for use in combination with the metal clip of FIG. 10(a) to secure the light source of FIG. 6 and 8;

FIG. 10(c) shows a plurality of LED's as conventionally fabricated on a die or chip; and

FIG. 10(d) show the LED used in the light source of FIG. 6 and 8 respectively.

### DETAILED DESCRIPTION

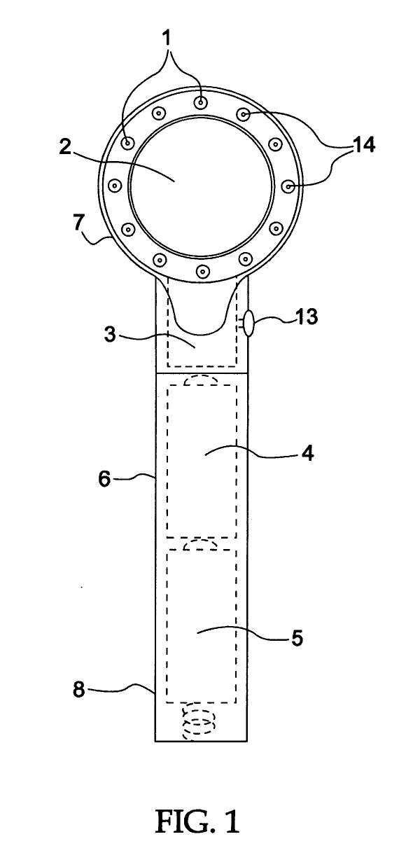

The optical screening device of the present invention is illustrated in FIGS. 1-4 represented by a simple hand held device 8 including a handle 6 and a head 7 extending from the handle 6. The head 7 defines a housing in which an array of light emitting diode's (LED's) 1 is mounted. A power source 4 or 5 for the array of light emitting diode's (LED's) 1, which may include one or more batteries, is preferably located in the handle 6. Light emitting diodes are low in cost and have lower power requirements due to their high efficiency. A single filter 2, preferably a high pass filter as is known to those skilled in the art or a bandpass filter, is supported by the device 8 at a location adjacent to the array of LED's 1 so that a substantial amount of the irradiated light reflected from skin tissue to be observed by the screening device 8 will primarily pass through the filter 2. Thus the filter simultaneously acts as a viewing area for the optical sensing device 8 as well as to block unwanted light energy of the primary excitation of the LED's.

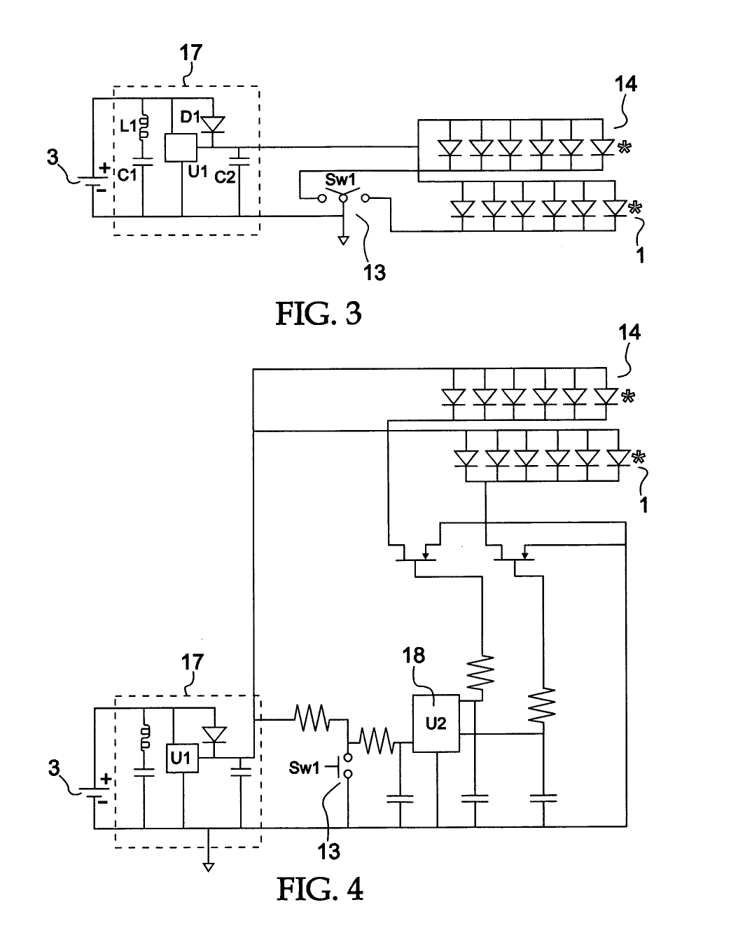

The arrays of LED's 1 are interconnected to one another to emit light in a wavelength range of between 340 NM to 470 NM when connected to a power source. The array of LED's 1 may be arranged to form any desired geometry but should preferably surround the filter 2 so that the area circumscribing the filter defines a fixed viewable window area for the screening device 8. The array of LED's 1 produce energy in a desired wavelength to provide a bioflourescent effect without the need for special power supplies, complex optics, heat blocking filters or light guides as in the prior art device. The power source for the array of LED's 1 can be a conventional power source such as a battery or plural batteries 4 and 5 as shown in FIG. 1 which may be connected to a control circuit 3 as shown in FIGS. 3 or 4 mounted in the handle part 6 of the device. The power source may also be a rechargeable battery or connected to an AC wall outlet. The handle 6 and head 7 may be composed of metal or of plastic.

In order to maintain constant light over a wide range of battery voltage or line fluctuation in the case of AC power, a voltage regulator circuit 17 may be included in the control circuit 3 for regulating the voltage of the power source.

Device Operation

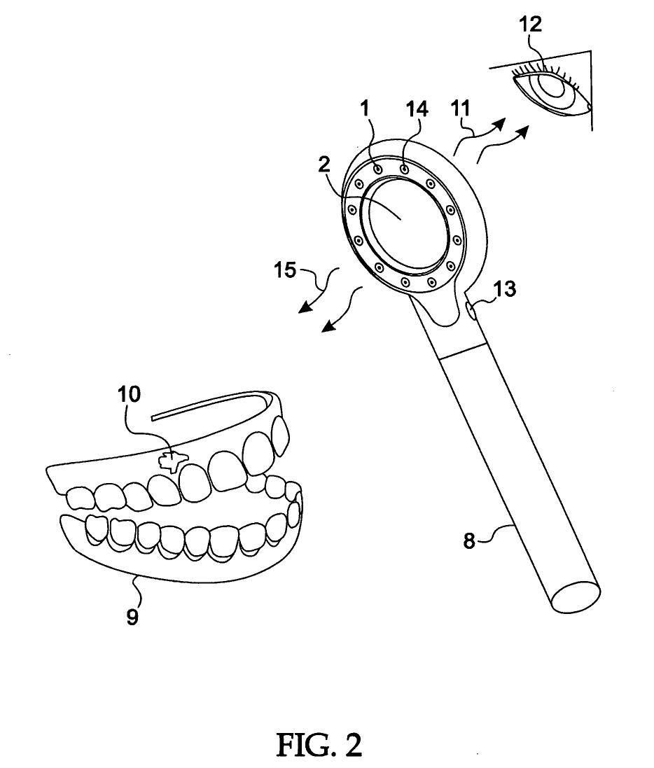

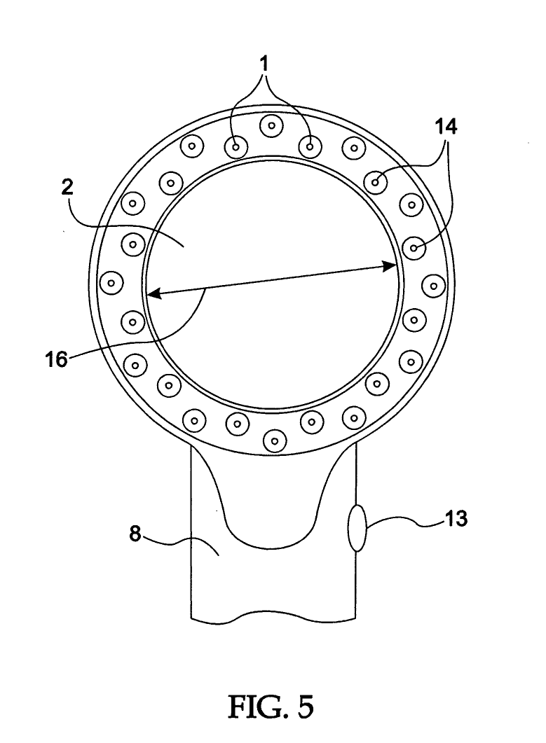

As shown in FIG. 2 the LED's 1 irradiate the mouth and associated oral structures which include the palate, tongue and oral mucosa 9. Irradiation may first be done with an array of LED's generating energy in the 340-400 NM range. A second examination using another or second array of LED's may then be made with energy in the 400-470 NM range. This can be accomplished with two devices 8 or using a single device 8 having a first and second array of LED's 1 mounted preferably in a concentric arrangement surrounding a filter 2 with each array of LED's consisting preferably of from 6 to 48 LED's as shown in FIG. 5. In the latter case a switch 13 is placed in the 340-400 NM mode and LED's 1 will illuminate 15 the suspected tissue 10. Next, a switch 13 is placed in the 400-470 NM mode and LED's 14 will illuminate 15 the suspected tissue 10. Both these wavelength spectrums will cause fluorescence of the surrounding healthy tissue and permit differentiation of a suspected lesion 10. Some lesions are observed better in one wavelength distribution or the other. In some cases it may be desirable to have both sets of LED's in the 340-400 NM range and the 400-470 NM range on simultaneously. In this condition the push button switch would scroll through this mode of operation as well. The operator 12 views the suspected lesions 10 through filter 2 and clearly sees its boundaries. This procedure may be carried out in a darkened room to increase contrast and eliminate ambient light. If more than one irradiating device 8 is used then one would be used to first examine in the range of between 340 to 400 nm and the other in the range between 400 nm to 470 nm. The filter 2 for the first irradiating device would preferably block light below 400 NM and the second would preferably block light below 470 NM. The examination would take place sequentially at the different excitation wavelengths.

The size of the filter 2 opening is very important. If the opening is too large then the irradiation of light from the circular array of LED's will land far outside the oral cavity. If the opening 2 is too small it will limit the viewable area of a suspected lesion and increase the time it will take to perform the oral examination. If the opening 2 is very small, the procedure becomes difficult and impractical, since the lesion itself might be larger than the viewable opening. For these reasons a device for oral examination should have a minimum opening diameter of ½ inch and a maximum diameter of 2½ inches. Preferably the opening 2 should be 1 to 2 inches in diameter.

FIG. 3 shows a drive circuit 16 which controls the functions of the LED's. The circuit contains a voltage regulator 17, which is also a dc to dc converter to provide constant output to the LED's. For example, using two 1.5 volt batteries for the battery source B, a total of 3 volts is available. The voltage regulator 17 will maintain a constant output voltage to the LED's of, e.g., even if the total battery voltage drops to 2.5 volts, providing longer useful battery life. Switch 13 is a multiposition switch. By depressing the switch in one position, LED array 1 is energized and in another position, LED array 14 is energized. In another mode of operation LED 1 and LED 14 are all on at the same time. Alternatively, a drive circuit is shown in FIG. 4, could also include a microprocessor controller, 18 which determines which LED's go on and the sequence of control i.e. LED's —OFF, LED array 1—ON, LED array 14—ON. The microprocessor 18 would also control additional LED arrays should that be desired.

It should be understood that although the filter 2 is preferably a high pass filter a band pass filter may be employed. Band pass filters are usually made by vacuum depositing many layers of metal oxide materials unto the glass. High and low pass filters are made by incorporating metal oxides into the glass during manufacturing process which is much less expensive. High pass filters used in this device can be made by vacuum deposition as well. Because the LED source provides a very narrow defined spectrum of energy, a high pass filter which is a much lower cost and is less prone to degradation may be used in our invention.

### Additional Embodiment

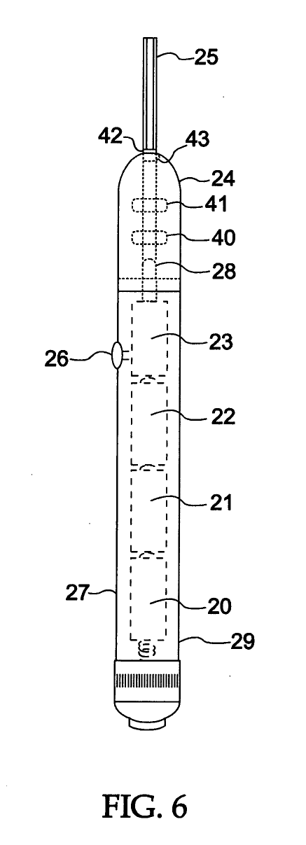

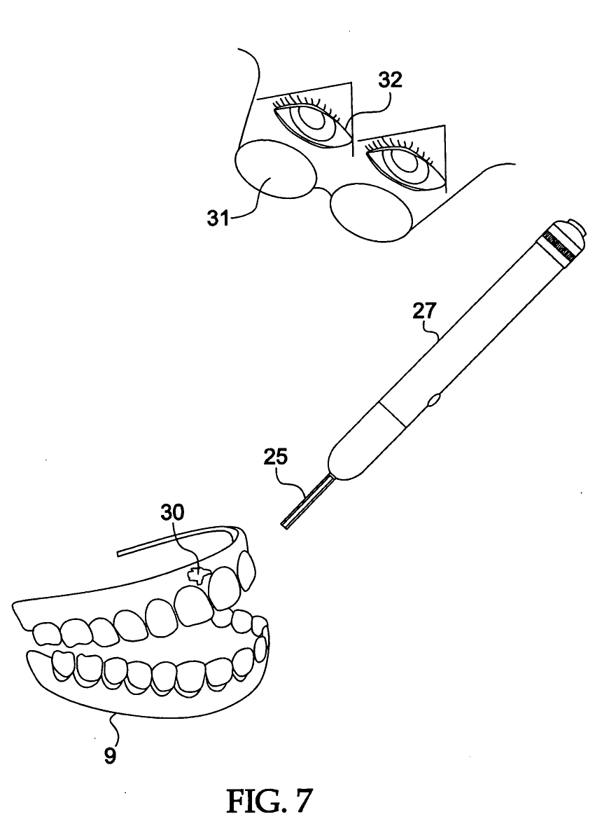

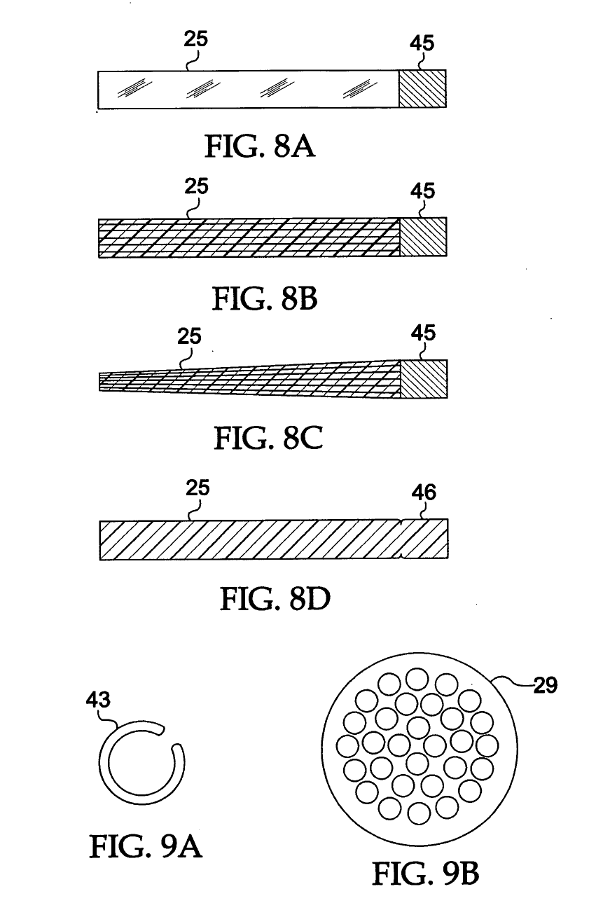

Another embodiment of the invention is shown in FIGS. 6 to 10 which comprises a light source 27 as shown in FIG. 6 and 8 in the shape and size of a relatively moderate length cigar having a hollow cylindrical handle 29 adapted to permit the light source 27 to be held in one hand. The handle 29 houses one or more batteries 20, 21, and 22 mounted in electrical series and in tandem in to one another. Alternatively, a conventional AC wall converter can be used and coupled directly to the handle to provide low voltage DC without using batteries. A nose cone 24 is removably connected to one end of the handle 29. One or more LED's 28 is mounted in the removable nose cone 24 and electrically coupled to the batteries 20, 21 and 22 in a series circuit when the LED's are of one wavelength range. One LED 28 or a plurality of LED's 28 are fabricated in a conventional manner on a single die or chip or on a plurality of chips and connected electrically in parallel to one another or in a series/parallel arrangement with respect to one another.

A manually operated switch 26 is mounted in the handle 29 and is electrically connected in a series circuit arrangement with the batteries 20, 21 and 22 and LED 28 for applying a voltage across the LED's of between 2.5 to 5 volts when the switch 26 is placed in the on position. A conventional light guide 25 is removably mounted to the nose cone 24 with one end adjacent to the LED's 28. Any conventional light guide 25 may be used preferably including a glass fused fiber light guide 25 or a tapered glass light guide 25 as shown in U.S. Pat. No. 5,371,826 the description of which is incorporated herein by reference or a clad glass rod or a transparent plastic rod 25. FIG. 25(a) shows a typical clad glass rod, 25(b) shows a typical glass rod made from image conduit, 25(c) shows a tapered glass rod made from image conduit, and 25(d) shows a plastic rod made from acrylic, polycarbonate, or other light conducting plastic. The holding means for securing the light guide 25 in the light source 27 consists of a metal clip ring 43 and a rubber “O” ring 44. The metal clip ring 43 is placed into one annular groove 41 and the “O” ring placed into another annular groove 42 built into nose cone 24 and form a locking mechanism for securing the light guide 25 in the nose cone 24 of the light source 27. The light guide 25 is inserted into the opening 42 of the nose cone 24 and the metal clip ring 43 snaps into place over the sleeve 45 of the light guide 25 or into a groove 46 when a plastic light guide as shown in 25(d) is used. The rubber “O” ring 44 acts to hold the light guide 25 so that it does not rotate in the light guide 25.

A voltage regulator circuit 23 of conventional design may also be housed in the handle 29 in circuit with the batteries 20, 21 and 22 and with the manual switch 26 to maintain a constant application of voltage across the LED's so that the light output will be relatively constant even with weak batteries. The switch 26 is manually operated to turn the light source 27 ON and OFF.

One or more LED's 28 operate in the 300 to 470 NM range. Multiple LED's may be used to cover currently available wavelength distributions. For example one set of LED's could operate from 340 to 400 NM while another set of LED's could operate from 400 to 470 NM. As explained earlier one or preferably a plurality of LED's 28 on multiple dies or chips of the same or different wavelengths can be employed. FIG. 10(a) shows the LED 28 and FIG. 10(b) is a magnified view of this LED with multiple LED's 28 on the same substrate.

The light source 27 is intended to be used with an external optical filter which is preferably fitted into or made part of a pair of eyeglasses 31 for the operator of the light source 27 to wear for use in conjunction with the light source 27 when viewing a suspected lesion. The light source 27 operates by turning the switch 26 to the on position and directing the light guide 25 inside an oral cavity such as the mouth of a patient. At the wavelengths described above the patient's healthy tissue will fluoresce, while unhealthy tissue, 30 will remain dark. The eyes 32 of the operator can view the tissue and for better contrast, the operator will wear a pair of eyeglasses 31 with high pass filters built therein. As described earlier, such filters are designed to block the primary beam of energy and allow fluorescent energy that is 40 to 60 NM in higher in wavelength to pass through.

1- An optical screening device for detecting abnormal skin tissue indicative of disease comprising:
a handle having an elongated stem adapted for holding the optical screening device in one hand and for placing the screening device into relatively close proximity to skin tissue for visual observation through the screening device;
a head connected to the elongated stem of the handle for housing an array of light emitting diodes with each diode in the array being spaced apart a predetermined distance to form an arrangement of more than at least three diodes in a given geometry with each diode selected to emit light in a wavelength range between 340 nm to 470 nm;
a high pass filter supported by said handle in close proximity to said array of light emitting diodes with the filter being of a size to provide a defined viewable area for visibly detecting irradiated light reflected from the skin tissue placed under observation by the screening device and adapted to block light at least below 470 nm; and a source of electrical power for energizing said array of light emitting diodes.

2- An optical screening device as defined in claim 1 wherein the skin tissue to be placed under observation is located in or about the oral cavity of the mouth.

3- An optical screening device as defined in claim 2 wherein the said array of light emitting diodes surrounds said high pass filter such that the defined viewable area is substantially circumscribed by said array of light emitting diodes.

4- An optical screening device as defined in claim 3 wherein the geometry formed by said array of light emitting diodes is circular and said viewable area is limited to about 2½ inches in diameter.

5- An optical screening device as defined in claim 4 wherein said array of light emitting diodes emits light in the 340 nm to 400 nm range.

6- An optical screening device as defined in claim 5 wherein said high pass filter blocks light below 400 nm.

7- An optical screening device as defined in claim 4 wherein said array of light emitting diodes emits light in the 400 nm to 470 nm range.

8- An optical screening device as defined in claim 7 wherein said high pass filter blocks light below 470 nm.

9- An optical screening device as defined in claim 2 wherein said array of light emitting diodes includes a first plurality of light emitting diodes for emitting light in a selected first wavelength range of between 340 nm to 400 nm and a second plurality of light emitting diodes for emitting light in a selected second wavelength range of between 400 nm to 470 nm.

10- An optical screening device as defined in claim 9 wherein said first plurality of light emitting diodes and said second plurality of light emitting diodes are arranged to substantially form concentric circles with said high pass filter surrounded by both said first and second plurality of light emitting diodes.

11- An optical screening device as defined in claim 10 further comprising switching means for selectively connecting said power source to said first or second plurality of light emitting diodes.

12- An optical screening device as defined in claim 11 wherein said switching means is a multipostion switch.

13- An optical screening device as defined in claim 10 further comprising a drive circuit for energizing said first plurality of first and said second light emitting diodes from said source of power, switching means for selectively connecting said power source to either said first or second plurality of light emitting diodes and a microprocessor for providing a controlled sequence for activating said drive circuit.

14- An optical screening device as defined in claim 13 wherein said switching means is a push button switch.

15- An optical screening device as defined in claim 13, where both a first and second plurality of LED's are on simultaneously.

16- An optical screening device for detecting abnormal skin tissue indicative of disease including a light source comprising a handle adapted to be held in one hand having at least one LED, a light guide, a source of low voltage DC for activating the LED in combination with an optical filter located external of the light source.

17- An optical screening device as claimed in claim 16 further comprising an eye glass holder or pair of glasses in which a pair of high pass filters are mounted.

18- An optical screening device as claimed in claim 17 wherein one end of the handle has a removable nose cone in which at least one LED is mounted.

19- An optical screening device as claimed in claim 18 wherein a plurality of LED's are mounted in the nose cone.
